(19) United States
(12) Patent Application Publication
(10) Pub. No.: US 2021/0252048 A1
(43) Pub. Date: Aug. 19, 2021

(54) TREATMENT OF LUNG AND AIRWAY DISEASES AND DISORDERS

(71) Applicant: AZ SOLUTIONS, LLC, Bloomfield Hills, MI (US)

(72) Inventor: Derek HILL, Bloomfield Hills, MI (US)

(73) Assignee: AZ SOLUTIONS, LLC, Bloomfield Hills, MI (US)

(21) Appl. No.: 17/307,232

(22) Filed: May 4, 2021

Related U.S. Application Data
(63) Continuation-in-part of application No. 16/641,749, filed on Feb. 25, 2020.
Application No. 16/641,749 is a continuation-in-part of application No. 15/686,612, filed on Aug. 25, 2017, now Pat. No. 10,960,129.
Application No. 15/686,612 is a continuation-in-part of application No. 15/686,617, filed on Aug. 25, 2017, now abandoned.
(60) Provisional application No. 62/660,670, filed on Apr. 20, 2018.
(60) Provisional application No. 63/019,741, filed on May 4, 2020.

(51) Int. Cl. A61K 33/30; A61K 9/12; A61P 31/16; A61K 31/375; A61K 45/06

(52) U.S. Cl. CPC A61K 33/30; A61K 9/12; A61K 45/06; A61K 31/375; A61P 31/16

(57) ABSTRACT

An aerosol containing fine droplets, the droplets separately or together containing a first component comprising an aqueous preparation of a transition metal or compound thereof and a second component which is an aqueous preparation containing at least one oxidation-reduction potential raising compound such as chlorite is useful in combating respiratory disorders and diseases caused by pathogens such as influenza, RSV, and coronavirus, as well as bacterial infections alone or associated with a viral pathogen.

### CROSS-REFERENCE TO RELATED APPLICATIONS

This application is a continuation-in-part of U.S. patent application Ser. No. 16/641,749, filed Feb. 25, 2020 (pending), which is the National Phase of PCT Application Serial No. PCT/US2018/048149 filed Aug. 27, 2018 (expired), which is a continuation-in-part of U.S. application Ser. No. 15/686,612, filed Aug. 25, 2017, now U.S. Pat. No. 10,960,129, issued Mar. 30, 2021, and is a continuation-in-part of U.S. application Ser. No. 15/686,617, filed Aug. 25, 2017 (abandoned) priority to both of which is claimed, and claims the benefit of U.S. Provisional Application Ser. No. 62/660,676, filed Apr. 20, 2018 (expired), and claims the benefit of U.S. Provisional Application Ser. No. 63/019,741 filed May 4, 2020 (pending), the disclosures of which are incorporated in its entirety by reference herein.

### 1. FIELD OF THE INVENTION

The present invention is directed to a method of treating lung and airway diseases and disorders with an at least two-component composition containing a transition metal in soluble form as a first treatment material, and a second component which contains an oxidizing agent which raises the oxidation-reduction potential of ions of the transition metal and of the exposed environment, and optionally further ingredients. The composition is introduced into the airways and/or lungs of a patient through aerosolization or nebulizing.

### 2. DESCRIPTION OF THE RELATED ART

Lung and airway diseases and disorders caused by pathogens are ubiquitous. In addition to the problems caused by the pathogens themselves and toxins associated therewith, there may be also inflammatory, reactive, allergic, irritative, restrictive, traumatic, constrictive, obstructive, and other tracheal, bronchial, bronchiolar or alveolar manifestations. These diseases and disorders are responsible for a great number of deaths worldwide, with an economic penalty measured in the billions of dollars. For example, according to WHO estimates, the global annual incidence of pneumonias is 450 million cases. Pneumonias lead to a total mortality of over 50 million people globally, and this is the number one cause of death in children, causing as many as 15% of child deaths worldwide. Of these pneumonias, viral pneumonia accounts for 13-50% of cases (approximately 200 million) as sole pathogens, and 8-27% of cases as mixed bacterial-viral infections. Several viruses in particular are implicated to be the causative agent, but the most common include influenzae, respiratory syncytial virus (RSV), and coronavirus. The worldwide pandemic occurring in 2020-2021 is evidence of the seriousness of such diseases. The economic burden of such diseases in the US, excluding those recently related to COVID-19, is already $17 billion annually.

Current management options for viral pneumonia include both preventatives/prophylactics and antiviral therapies. Prophylactic therapies are limited in their availability, effectiveness, and lack of cross-reactivity/protection against non-matching strains or vaccine escape mutations. Up until recently, influenza was the leading organism involved in viral pneumonia, and a few antiviral medications have been proposed for its treatment. Treatment options for viral pneumonias caused by influenza include oseltamivir, baloxavir, marboxil, permivir, and zanamivir. Those caused by RSV and coronavirus can be treated using ribarvirin and remdesivir, respectively. With no known highly efficacious direct-acting treatments for pneumonia caused by RSV and coronavirus, standard care is largely supportive. While all these antivirals have the potential to reduce median time to resolution of viral infections, they are susceptible to the development of resistance. Additionally, antibiotics are used to exclude or treat concomitant bacterial infection, but they cannot treat viral infections. These antibiotics are also susceptible to development of resistance by the bacteria they are meant to treat. There is a significant need for novel treatments with greater potency that do not contribute to resistance.

### SUMMARY OF THE INVENTION

The inventor has surprisingly and unexpectedly discovered that the use of a two component therapeutic solution, one containing transition metal ions, and the second component containing a substance which raises the oxidation-reduction potential, can be aerosolized or nebulized and introduced in this form into the lungs and airways of a patient suffering from one of the above-described diseases or disorders. This composition actively combats both viral and bacterial infections, and also eradicates biofilms of both Gram-positive and Gram-negative organisms, which can be useful in eradicating pneumonias, including those caused by multi-drug-resistant bacteria, and in clearing colonization of the lungs and airways by bacteria or other pathogens in patients with cystic fibrosis or other lung diseases.

### DETAILED DESCRIPTION OF THE PREFERRED EMBODIMENTS

The therapeutic components of the invention are aerosolized or nebulized for use as an inhalational therapeutic. The terms “aerosolized” and “nebulized” are intended to mean that the liquid components of the invention are converted into very fine droplets, preferably with mean droplets sizes of less than 3 μm, such that these can be inhaled easily, and can penetrate into the alveoli of the lungs. By forming such small droplets, effective exposure to viral pneumonia pathogens in the recesses of the lungs is enabled. Hereinafter, the terms “aerosolization” and “aerosolized” will be used to describe the nature of the composition to be inhaled, regardless of its means of preparation. Other synonyms included by the term “aerosolization” and like terms, are atomization, misting, evaporation, spraying, irrigation, ventilation and volatilization. The term “aerosolization” and similar terms are not meant to be restrictive.

The first component of the two component therapeutic solution contains at least one transition metal ion which is physiologically tolerated by a human or other mammalian subject. There are several transition metals which are known to be efficacious in this respect, in particular zinc ions, but also ions of other transition elements such as silver, copper, cobalt, and nickel, among others. While the ionic form of each of these elements is preferable, in some cases these elements may also be supplied in elemental form or in the form of non-ionic compounds. For example, the transition metal compound may be supplied dissolved or dispersed in a liquid, preferably water. What is important in each case, is that the elements, however supplied, can be converted into ionic form in vitro prior to aerosolization, or in vivo, post inhalation. Complete conversion to an ionic form is not necessary.

Examples of zinc-containing compounds which are suitable in aqueous solution for the first component of the two component therapeutic solution include, but are not limited to, zinc chloride, zinc sulfate, zinc iodide, zinc fluoride, zinc phosphate, zinc bromide, zinc acetate, zinc oxide, zinc citrate, zinc lactate, zinc nitrate, and zinc salicylate. Some of these zinc-containing compounds as well as some of the compounds of other transition metals may not be soluble in water as such, but can frequently be converted to a water-soluble form by altering the pH of the aqueous solution, as is well known to those skilled in the art. For example, zinc oxide can be solubilized in water through addition of an acid such as hydrochloric acid.

Examples of silver-containing compounds include, but are not limited to, silver chloride, silver fluoride, silver difluoride, silver hydroxide, silver bromide, silver iodide, silver sulfate, silver sulfide, silver acetate, silver oxide, silver citrate, silver carbonate, silver lactate, silver nitrate, silver salicylate, silver phosphate, silver diamine fluoride, silver sulfadiazine, and silver benzoate.

Copper-containing compounds include, but are not limited to, copper chloride, copper chlorate, copper carbonate, copper fluoride, copper hydride, bromide, copper iodide, copper sulfide, copper sulfate, copper hydroxide, copper silicide, copper acetate, copper oxide, copper citrate, copper lactate, copper nitrate, copper salicylate, and copper benzoate.

Cobalt-containing compounds include, but are not limited to, cobalt chloride, cobalt chlorate, cobalt fluoride, cobalt bromide, cobalt iodide, cobalt sulfate, cobalt sulfide, cobalt phosphate, cobalt selenide, cobalt phosphide, cobalt cyanide, cobalt carbonate, cobalt oxalate, cobalt acetate, cobalt oxide, cobalt hydroxide, cobalt citrate, cobalt hydride, cobalt chloride, cobalt lactate, cobalt nitrate, cobalt salicylate, cobalt benzoate, cobalt blue, and cobalt thiocyanate.

Nickel-containing compounds include but are not limited to nickel chloride, nickel hydroxide, nickel fluorides, nickel bromide, nickel iodide, nickel sulfate, nickel cyanide, nickel carbonate, nickel phosphate, nickel acetate, nickel chloride, nickel silicide, nickel carbide, nickel oxide, nickel citrate, nickel lactate, nickel nitrate, nickel salicylate, and nickel benzoate.

It is preferable that the transition metal or compound or complex thereof be at least partially soluble in aqueous solution, most preferably completely soluble in the concentration used. It is frequently preferable to alter the pH of the solution/dispersion. An acid pH has been found to be preferable in many cases, especially a pH from about 4.0 to <7.0. More acidic solutions/dispersions can often be employed when the pH of the second component is basic.

The transition metals used may be one type of transition metal or more than one type of transition metal. For example, in some biocidal applications, mixtures of copper ions and silver ions have proven to be particularly effective. However, two component compositions wherein the transition metal-containing component contains only a single transition metal are preferred.

The concentration of transition metal in the first component may range from 0.001 moles per liter to about 0.25 moles per liter. Concentrations higher and lower than these amounts may be used depending upon the circumstances. For example, the aerosolization of the first, the second, or both the first and second components of the two component therapeutic composition may be accompanied by aerosolization of other components, such as water or physiological saline. In such a case, a higher concentration of transition metal may be utilized. The upper limit of concentration of transition metal in the aerosol may be determined by conventional methodology, for example by utilizing Student's t-test to compare treated tissues versus saline-exposed controls. Minimum concentrations required for inhibition of 50% and 90% of pathogen, the 50% and 90% effective concentrations (EC50 and EC90) can be readily determined, for example by in vitro 3-D alveolar and lung epithelial tissue constructs with infected and noninfected tissues. These values can be compared to saline-exposed controls in infected and noninfected tissues. The influenza virus H1N1 is suitable for such studies. Additional studies with RSV and human coronavirus can also be easily performed. Each exposure to the activated transition metal solution is followed by histologic examination at critical points. These critical points will be triggered by statistically significant increases in lactate dehydrogenase (LDH) and inflammatory markers interleukin-1 (IL1) and interleukin-6 (IL6). Histologic examination interpretation can be conducted based on the Position Paper of the Society of Toxicological Pathology (2013).

Once the effective concentrations are determined, the concentration of which there is 50% toxicity of the tissues being tested (TD50) is also determined. The dose can be calculated to produce the desired therapeutic response, the therapeutic index, by the quotient of TD50/EC50. This methodology is well known to those skilled in the art.

The second component of the two component therapeutic solution contains a substance which raises the oxidation-reduction potential such that the transition metal will be in ionic form. The second component generally contains an oxidizing agent, this oxidizing agent being physiologically tolerable by a human or other mammalian species. Examples of such oxidizing agents are chlorite ions and hydrogen peroxide. Chlorite ions, preferably supplied as an alkali metal chlorite, are preferable. Many transition metal compounds, though soluble in water, do not exist in water in activated ionic state. One of these, for example, is zinc chloride, which does not exist in aqueous solution solely as zinc(II) ions and chloride ions, but rather, without the oxidation-reduction second component of the invention, would exist in the form of a variety of soluble or complexed multi-atom species containing, for example, zinc, oxygen, hydroxide ions, chloride ions, and the like. The use of the claimed second component increases the oxidation-reduction potential and maximizes the amount of transition metal ion which is in a highly ionized state.

The concentration of the second component can vary over wide limits, but is generally from 0.01 mole/liter to about 10 mole/liter, more preferably from 0.02 mole/liter to 0.2 mole/liter. The ratio of the oxidation-reduction potential raising substance, or “oxidation-reduction enhancer” to transition metal or compound or complex thereof, calculated as equivalents, is preferably from 0.1 to 10, more preferably 0.5 to 2. The amounts may be easily and routinely determined based on the test previously described relative to the transition metal.

Preferred compositions further contain ascorbic acid or citric acid, preferably ascorbic acid. The ascorbic acid or citric acid is preferably contained in the transition metal ion-containing component (component A in the tests described later), but in principle can also be present all or in part in the second (B) component, or as a third (c) component.

Metabolism and growth of pathogens may increase when an oxidation-reduction potential (Eₕ) is decreased. The Eₕ in such areas can decrease, for example, when the oxygen decreases, through bacterial mechanisms, or in response to other changes in the characteristics of a patient. Conversely, by increasing the Eₕ, metabolism and growth of pathogens may be decreased, which may prevent, decrease and/or cure an infection. In certain instances, the metabolism and growth of the microorganisms may be prevented by increasing the oxidation-reduction (redox) potential (or Eₕ) of the patient's tissue by contact with an aerosol containing the oxidation-reduction potential enhancer. Furthermore, such contact may help eradicate and/or prevent the formation of biofilms. Biofilms are a microbial resistance to host defenses and antibiotics and antivirals, and preventing or eradicating their growth or production can eradicate or eliminate microorganism infection, inoculation, colonization or other pathology.

Increasing the Eₕ in the lungs and/or airways may include introducing one or more electron-rich or ion-rich compounds, such as a charged zinc compound having free available zinc ions and at least one Eₕ raising component, in the form of an aerosol. Accordingly, a system that charges a charged compound (or charged fluid) and provides the charged compound to the patient's lungs and/or airways is desirable.

According to one or more embodiments of the present disclosure, to increase the redox potential of a patient's lungs and/or airways, one or more electron-rich or ion-rich compounds, such as ionically charged chemical compositions (e.g., zinc, copper, or silver) having free available ions, and at least one redox potential raising component, may be supplied as an aqueous aerosol. Solutions suitable for producing such aerosols and increasing the redox potential for infection prevention/control/curing include, but are not limited to, solutions forming Zn²⁺, Ag⁺or Cu²⁺ ions, or solutions with any combination of zinc, copper, and silver ions. In certain embodiments, the solutions include salts of a weak acid to provide the free available ions in the charged compound. In at least one embodiment, the weak acid is soluble in water. Examples of zinc compounds that form solutions with free available zinc ions include, but are not limited to, zinc chloride, zinc acetate, zinc lactate, zinc salicylate, zinc sulfate, zinc nitrate, zinc stearate, zinc gluconate, zinc ammonium sulfate, zinc chromate, zinc citrate, zinc dithionate, zinc fluorosilicate, zinc tartrate, zinc formate, zinc iodide, zinc phenol sulfonate, zinc succinate, zinc glycerophosphate, or other zinc halides. Examples of copper compounds that form solutions with free available copper ions include, but are not limited to copper acetate, copper formate, copper citrate, copper lactate, copper oxalate, copper propionate, copper benzoate, copper succinate, copper malonate, copper stearate, copper gluconate, copper ammonium sulfate, copper chromate, copper dithionate, copper fluorosilicate, copper tartrate, copper iodide, copper nitrate, copper phenol sulfonate, copper salicylate, copper sulfate, copper glycerophosphate, or other copper halides. Examples of silver compounds that form solutions with free available silver ions include, but are not limited to silver sulfate, silver oxide, silver acetate, silver nitrate, silver citrate, silver chloride, silver lactate, silver phosphate, silver stearate, silver thiocyanate, silver saccharinate, silver anthranilate, silver carbonate, or other silver halides. The solutions providing the ions, or mixed with such solution, may have preferred solubility for the specific ion selected, as well as provide an effective amount of ions without permitting the redox potential to fall to unwanted levels. The aerosol formed by the combining the solutions may be referred to as a “charged aerosol,” “charged fluid,” “ionically charged fluid,” or “charged compound.” For example, to form zinc ions, ZnCl₂ and NaClO₂ may be combined at the time of administration to the tissues as two solutions to form the charged active component. The two solutions may be combined in a charging area to form a charged compound having free available ions and at least one redox-potential-raising component (e.g., the NaClO₂ is a source of chlorite ions to raise the redox potential while also interacting with the ZnCl₂ to form zinc ions).

In some embodiments according to the principles of the present disclosure, an ion-rich or electron-rich compound suitable for treating lung pathogens can be formed by combining a first fluid component and a second fluid component in a “charging area.” For example, in some embodiments, a system can include a fluid container, such as one or more intravenous bags hung from an intravenous stand. The fluid container includes a first fluid compartment and a second fluid compartment, or the two fluids could be held in separate containers. In some embodiments, a first component, such as aqueous zinc chloride (ZnCl₂), is retained in the first fluid compartment and a second fluid, such as aqueous sodium chlorite (NaClO₂), a source of chlorite ions, is retained in the second fluid compartment. Before use, a portion of the first fluid and a portion of the second fluid are released from the first fluid and second fluid compartments, respectively, into a “fluid charging portion” or “mixing area.” Mixing may not involve agitation, or mixing devices can be used, e.g. a static mixer. The portion of the first fluid and the portion of the second fluid mix and/or interact in the fluid charging portion to form a “charged compound.” For example, a portion of the ZnCl₂ interacts with a portion of the NaClO₂ to form a charged zinc compound having free available zinc ions and at least one Eₕ raising component (e.g., the NaClO₂ acts like an Eₕ raising component with the chlorite ions and when interacting with the ZnCl₂). The composition is then aerosolized. By providing the charged compound to a patient in the form of an aerosol, the free available zinc ions and the at least one Eₕ raising component has been shown to prevent lowering of the Eₕ levels, thereby inhibiting metabolism and/or growth of bacteria, which may prevent and/or cure infections.

Although sodium chlorite is referred to herein, it should be understood that other redox potential-raising compounds as a source of stable chlorite, such as oxychlorides, which can be prevented from degrading to chlorine dioxide, are contemplated by the present disclosure, and the embodiments with sodium chlorite are described as examples. The chlorine dioxide of the NaClO₂ (sodium chlorite) has antimicrobial properties and has been used for disinfection and control of bacterial fouling, as well as controlling taste, odor, and oxidation of metal ions. Using sodium chlorite as a redox potential-raising compound rather than as a source of chlorine dioxide is very important, because chlorine dioxide at elevated levels may combine with amino acids to produce potentially mutagenic compounds. Chlorine dioxide is made available at lower, controlled levels by maintaining a neutral or basic pH of the component providing NaClO₂, such that degradation of the sodium chlorite is avoided. Furthermore, neutral pH or basic pH provides chlorite ion stability. Additional components, such as, but not limited to, hydrogen peroxide, may be included in the composition to help stabilize the ions, and control pH of the separate solutions, and of the charged compound.

Furthermore, the charged compound may further include an antibiotic agent, antiviral agent, antifungal agent, other antimicrobial agent, or combinations thereof, in either or both solutions prior to combining to form the charged wound irrigant, or include the antibiotic agent, antiviral agent, antifungal agent, other antimicrobial agent, or combinations thereof in a separate solution for adding to the charged wound irrigant. In some embodiments, the charged wound irrigant includes one or more antibiotic agent, antiviral agent, antifungal agent, other antimicrobial agent, or combinations thereof. The antibiotic agent, antiviral agent, antifungal agent, other antimicrobial agent, or combinations thereof, may be selected per the type of infection. The charged wound compound may also include a protein such as a transforming growth factor beta (TGF-β) protein to promote healing. The TGF-β additive may be any of three isoforms of TGF-β (β1, β2, β3) or a combination thereof.

The solutions forming the aerosol may be separately stored until time for use, as the charged nature of the aerosol components may be temporary. That is, the charged wound compound may lose its charge over time as the free available ions are reduced such that it may be unable to raise the redox potential of the composition (or inhibit lowering of the redox potential). The solutions are stored separately at either neutral, acidic or basic pH, and combined at the time of use. For example, storing sodium chlorite at neutral or basic pH may prevent any significant degradation to chlorine dioxide. Storing of zinc chloride at acidic pH may help insure the availability of zinc ions. Also, an acidic pH may convert sulfur anions to the acidic forms which result in a higher Eₕ. In the case of hydrogen sulfide, because hydrogen sulfide is volatile, its formation serves as an effective means of getting rid of electrons carried by sulfide anion that are particularly conducive to lowering the Eₕ. Third, catalase degradation of hydrogen peroxide may be inhibited at an acidic pH. An acidic pH and the presence of chloride ion ensures that the hydrogen peroxide in the composition is not degraded with storage and hence retains its effectiveness.

Based on the preliminary data against human coronavirus and H1N1 influenza virus, as well as Methicillin-Resistant Staph Aureus and Pseudomonas aeruginosa biofilms, this technology would be an attractive candidate for combating other types of infectious lung pathologies such as cystic fibrosis. Preliminary data demonstrated 5-fold better in vitro efficacy against S. aureus and P. aeruginosa biofilms compared to existing antimicrobial solutions and in vivo antimicrobial efficacy in a pig study.

There is a significant paucity of treatments for viral pneumonias. There are currently one inhaled and 2 oral medications for influenza, and the efficacy of anti-influenza medications is limited. In fact, some of the efficacy data that has been relied upon has recently been called into question, and treatment of other viral pneumonias is essentially supportive. The revolutionary approach described herein will add significant tools to this limited armamentarium.

Preliminary research has shown that the individual active components of the claimed invention have potent bactericidal, including anti-biofilm, properties on their own. Together, these active components work synergistically in their antimicrobial activity. Moreover, potent antiviral activity has been demonstrated as well. The components are soluble in aqueous solution and can be easily nebulized or aerosolized to directly treat airways, lung parenchyma and alveoli without compromising efficacy. Mucous often presents a barrier to treatment, and the anti-biofilm capability of our components makes them mucolytic candidates as well.

The claimed invention recruits the antiviral properties of certain transition metals for novel use in an inhalational agent. These metals, preferably in millimolar concentrations, can be both nontoxic and efficacious against a broad spectrum of viruses. Zinc, in particular, plays an important part in immune system function and is an important cofactor in many eukaryotic cellular processes. Zinc is an important component of Zinc-finger Antiviral Proteins, which are expressed in lung cells and significantly restrict SARS-CoV-2. Zinc upregulates expression of IL-2, which stimulates natural killer production and T cells to kill viruses and bacteria, and IFN-γ, which activates macrophages to kill parasites. Zinc also suppresses ICAM-1, which serves as a receptor for viruses, and inhibits the protease from HIV type 1.

The efficacy of zinc is improved when in its ionic, “activated” state. In this state zinc interferes with viral replication via free virus inactivation, inhibition of viral uncoating, viral genome transcription, and viral protein translation and polyprotein processing.

Depending on the source compound of zinc, pH of the solution, buffers, etc., the degree of zinc ionization can vary considerably. In the claimed invention, zinc chloride (ZnCl₂) is preferred for its high solubility and potential ionic availability. Mixing a transition metal compound with an oxidizing agent can result in a highly ionized state for the metal, as the oxidizing agent draws electrons away from the transition metal ions. The choice of oxidizing agent for this task, sodium chlorite (NaClO₂), can significantly raise the oxidation-reduction potential of its environment and adds its own significant antimicrobial properties to the composition. The two components which are generally recognized as safe (GRAS) work synergistically to produce a potent antiviral combination potentially suitable for exposure to sensitive lung tissue.

Because of the high reactivity of the two-component system, it is necessary to store the ZnCl₂ and NaClO₂ solutions separately. The ZnCl₂ is more stable, and therefore stored, in an aqueous solution at acidic pH to help prevent association with negatively charged ions or precipitation, maintaining it in a more ionic, +2 state. NaClO₂ is stable, and therefore stored, at more basic pH. When the two are combined (with a combined acidic pH) the ionized zinc is eluted from the ZnCl₂, and oxygen free radicals are created from the NaClO₂. Both of these processes are transient, so the key to the claimed invention is that the components are stored separately and mixed immediately before use.

The claimed invention is innovative because there are currently no inhaled antivirals in use other than zanamivir, which is ineffective against viruses other than influenza. Beyond this, often more than one organism, bacterial and/or viral, is involved in bronchopulmonary infectious disease. For this reason there is a need for a ubiquitous antiviral; in particular, one which also has antibacterial properties. Importantly, because the claimed aerosols are rapidly cidal, they are not susceptible to bacterial or viral mutation or antibiotic/antiviral resistance.

Compositions of the claimed invention have shown clear in vitro efficacy at one-hour and 4-hour exposure times against Coronavirus strain OC43 and Influenza A H1N1 strain A/WS/33 in studies based on ASTM-E1052-11 Standard Test Method to Assess the Activity of Microbicides against Viruses in Suspension. Demonstration of >4-log and >5-log eradication of the 2 viruses, respectively, were shown.

Extensive testing has also been performed on bacterial biofilm eradication in vitro. through multiple studies using a Modified Robbins Device, as well as infected endotracheal tubes. A >6.5-log reduction (100% eradication) of Methicillin-resistant Staph Aureus (MRSA) biofilms after 2-hour exposure has been shown, as well as a similar reduction (>5.5-log) in Pseudomonas aeruginosa biofilms after 2-hour exposure. The anti-biofilm data is important to the current application because ability to penetrate biofilm could translate to ability to penetrate mucous and eradicate pathogens sequestered from other antimicrobial treatments. Stability testing of the current invention after one year without special storage conditions has demonstrated >4-log reduction of Pseudomonas biofilms after 2-hour exposure.

A preliminary safety study was also performed while testing for antibacterial properties of the solution in vivo in a pig wound model. After inoculation of 24 wounds on the back of a single pig with Pseudomonas aeruginosa and Staph aureus, the wounds were treated with our activated zinc solution. After 24-hours exposure, the wounds were placed in formalin for pathologic examination. There was found to be no statistically significant tissue necrosis by a blinded veterinary pathologist after this prolonged exposure. Importantly, the efficacy against Staph aureus and Pseudomonas was preserved in this in vivo model where many antimicrobials fail.

Preliminary studies have shown clear in vivo efficacy and safety of our zinc-based solution in a pig wound model. They have also shown clear efficacy against 2 important respiratory virus analogs in vitro. Tissue-specific efficacy and toxicity studies to determine Therapeutic Index, as well as development of an aerosolized delivery system through in vivo animal models for efficacy and safety must proceed before human clinical trials.

In order to be effective against pneumonia, inhalational antimicrobials need to be able to reach the alveoli of the lungs. For this to occur, droplet sizes need to be in a range preferably below 3 μm (mean droplet size) more preferably in the range of 1-3 microns. Droplets larger than this adsorb to the airways and cilia. Methods of aerosolization are well known in the art, and include Venturi spray devices, ultrasonic aerosolization devices, and other devices, for example those employed today in electronic vaping apparatuses. The particular method employed is not important, so long as the particle size of the aerosolized droplets is very small, preferably in the range of 3 μm or less. Particle size and particle size distribution of the aerosolized droplets can be measured by standard methods.

In addition to the two components just described, either component or a further component may also contain additional ingredients to aid in treating the particular disorder or disease. These additional components include antimicrobials, both bacterial antimicrobials and antivirals, vitamins, especially vitamins such as vitamins A, C, B and D, desensitizing agents such as lidocaine, Novocaine, and the like, as well as NSAIDs and steroidal desensitizing agents such as prednisone and methylprednisone, beta agonists such as albuterol, terbutaline, salmeterol, levalbuterol, pirbuterol, formoterol, indacaterol, olodaterol, and vilanterol, and pain killers such as oxycodone and morphine and fentanyl in therapeutically effective and tolerated amounts. The components, either or both, may also contain electrolytes, including saline, but also, by way of example, magnesium, calcium, potassium and other electrolytes. The antivirals, antibacterials, vitamins, analgesics, NSAIDs, steroids, beta agonists, and pain killers such as oxycodone, morphine, fentanyl, and the like, somniferants and desensitizing agents may be considered “active substances” as that term is used herein, while electrolytes and similar ingredients are best termed “adjuvants” herein. This list of additives and adjuvants is not limiting.

The present invention is principally directed to a method for treating a patient having a lung or breathing system disease or disorder involving a microbial pathogen. In this method, the two principal components of the present invention are aerosolized separately or from a common solution, preferably one which is formed from two separate components combined just prior to aerosolization, optionally in conjunction with other components as mentioned above, are introduced into the lungs, trachea, or other breathing passages in the form of an aerosol, preferably an aerosol having mean particle sizes below 3 μm, preferably below about 2 μm, and most preferably in the range of 0.5 μm to 2 μm, incrementally or continuously. The introduction may be by a separate device, or the aerosol may be introduced into an oxygen tube, breathing tube, or the like which is already in place. Introduction may be continuous, e.g. for periods of 10 minutes to several hours, even days, or may be intermittent. Intermittent use is expected to be well tolerated in mammalian species, particularly humans.

The invention has the advantage that the means for combating the pathogens is one which the pathogen is extremely unlikely to be able to mutate to avoid. It has the further advantage that the ingredients are far less expensive than many of the remedies currently being employed, and the still further advantage that the transition metal ions in their activated state are effective over a wide range of pathogens as opposed to being effective on only particular pathogens.

A study was made to evaluate the virucidal capacity of one test product versus three viruses. The study design was based on ASTM El 052-11 Standard Test Method to Assess the Activity of Microbicides against Viruses in Suspension. All testing was performed in accordance with Good Laboratory Practices, as specified in 21 CFR Part 58. Under the conditions of this evaluation the Test Product, the combination of Solution A containing zinc ions and Solution B containing sodium chlorite, reduced the infectivity of Coronavirus strain OC43 by >4.00 log 10 (>99.99%) following both exposure times (1-hour and 4-hours). Similarly, the combination of Solution A and Solution B, reduced the infectivity of influenza A HINT strain A/WS/33 by >5.00 log₁₀ (>99.99%) following the 1-hour exposure time and by >4.75 log₁₀ (>99.99%) following the 4-hour exposure time. Furthermore, the combination of Solution A and Solution B, reduced the infectivity of Simian virus 40 (SV-40) by 0.25 log₁₀ (43.77%) following the 1-hour exposure time and by 2.75 log₁₀ (99.82%) following the 4-hour exposure time.

The virucidal capacity of the claimed invention versus three viruses was evaluated. The study design was based on ASTM E1052-11 Standard Test Method to Assess the Activity of Microbicides against Viruses in Suspension. All testing was performed in accordance with Good Laboratory Practices, as specified in 21 CFR Part 58.

The study was designed to evaluate whether the claimed combination could inactivate each of the three challenge viruses using a Virucidal Suspension Test (In-Vitro Time-Kill method). Challenge viruses included human Coronavirus strain OC43 (Betacoronavirus), Influenza A H1N1, and Simian virus 40 (SV-40) (Human Papilloma Virus surrogate). The percent and log₁₀ reductions from the initial population of the viral strains were determined following exposure to the test product for 1 hour and 4 hours. Plating was performed in four replicates.

The Study Protocol, reproduced immediately below, presents the study methodology, in detail.

Host Cell Preparation:

Cells, obtained from American Type Culture Collection (ATCC), are maintained as monolayers in disposable cell culture labware in accordance with BSLI SOP L-2084, “Procedure for Subculturing of Cells.” Prior to testing, host cell cultures are seeded onto 24-well cell culture treated plates. Cell monolayers are more than 48-hours old and 80-90% confluent before inoculation with the virus. The growth medium (GM) and maintenance medium (MM) will be RPMI-1640, MEM, or EMEM, as appropriate for each cell culture.

| | | Growth | |
|---|---|---|---|
| | Host | Medium | Maintenance Medium |
| Virus | Cell | Components | Components |
| Coronavirus | HCT-8 | RPMI-1640 | RPMI-1640 |
| strain OC43 | | 10% Horse | 2% Horse Serum |
| | | Serum | 1% Antibiotic |
| | | 1% Antibiotic | |
| Influenza A H1N1 | MDCK | MEM | MEM |
| | | 10% FBS | 1% Antibiotic |
| | | 1% Antibiotic | 1% L-glutamine |
| | | 1% L- | ~1 μg/mL TPCK |
| | | glutamine | treated Trypsin |
| SV-40 | BS-C-1 | EMEM | EMEM |
| | | 10% FBS | 2% FBS |
| | | 1% Antibiotic | 1% Antibiotic |

Test Virus Preparation:

Virus propagated and stored per BSLI SOP L-2102, Procedure for Production of High-Titered Virus Stock, is used for this study. On the day of use, aliquots of a stock virus suspension are removed from a −70° C. freezer and thawed.

Virucidal Suspension Test:

The Virucidal Suspension Test will include the following parameters:

| | | Plating |
|---|---|---|
| Parameter | Summary | Replicates |
| Virucidal | 1 Part Virus + 9 Parts Test Product Exposure Neutralization | 4 per dilution |
| Suspension Test | Dilution Plating | |
| Virus Control | 1 Part Virus + 9 Parts Diluent Exposure Dilution Plating | 4 per dilution |
| Cytotoxicity | Diluent + Test Product Neutralization Dilution Plating | 4 per dilution |
| Control | | |
| Neutralization | 1 Part Diluent + 9 Parts Test Product Neutralization | 4 per dilution |
| Control | Virus inoculation Dilution Plating | |
| Neutralizer Toxicity | Virus + Diluent Neutralization Dilution Plating | 4 per dilution |
| Control | | |
| Cell Culture | Maintenance Medium | Minimum4 |
| Control | | wells |

Test: A 0.5 mL aliquot of test virus(s) is added to a vial containing 4.5 mL of the undiluted test product to achieve a 90% (v/v) concentration of the Test Product. The test virus(s) are exposed to the test product for 1 hour and 4 hours, timed using a calibrated minute/second timer. The calibrated minute/second timer is started within ±1 second of adding the challenge suspension. Immediately after each exposure, the test virus(s)/test product suspensions are neutralized in appropriate neutralizer, mixed thoroughly, and serially diluted in MM. Dilutions will be plated in replicates of four wells.

Virus Controls (1-hour and 4-hour): A 0.5 mL aliquot of test virus(s) is added to 4.5 mL of MM and exposed for 4 hours at ambient temperature. The subsequent test virus dilution is made in MM and serially diluted in MM. Dilutions will be plated in replicates of four wells upon elapse of 1-hour exposure and 4-hour exposure.

Cytotoxicity Control: A 0.5 mL aliquot of MM is added to a tube containing 4.5 mL of the undiluted test concentration. The MM/product mixture is neutralized in appropriate neutralizer, mixed thoroughly and serially diluted in MM. Dilutions are plated in replicates of four wells.

Neutralization Control: A 0.5 mL aliquot of MM is added to a tube containing 4.5 mL of the undiluted test product. The MM/product mixture is diluted 1:10 in an appropriate neutralizer. An aliquot of the neutralized product is discarded and replaced with an aliquot of the virus(s), thoroughly mixed, and exposed for 10 to 20 minutes. Subsequent 10-fold dilutions of neutralized test product/MM are made in MM. Dilutions are plated in replicates of four wells. The Neutralization Control is plated after the 1-hour Virus Control has been plated.

Neutralization Toxicity Control: Additionally, the effect of the neutralizer on virus infectivity is assessed by adding 0.5 mL of virus to 4.5 mL of neutralizer (appropriate neutralizer) alone followed by exposure for 10 to 20 minutes. Subsequent 10-fold dilutions of virus suspension/neutralizer is made in MM. Dilutions are plated in replicates of four wells. The Neutralization Toxicity Control is plated after the 1-hour Virus Control has been plated.

Cell Culture Control: Intact cell culture serves as the control for cell culture viability. The GM will be replaced by MM in all cell culture control wells.

All exposures occur at ambient room temperature (18-25° C.). Ambient temperature and humidity will be recorded during testing.

The plates are incubated in a CO₂ incubator for approximately 3 to 7 days at 35° C.±2° C. (Influenza A) and approximately 10 to 21 days at 33° C.±2° C. (Coronavirus strain OC43), and for approximately 10-21 days at 37° C.±2° C. (SV-40) in CO₂ incubator(s) or for the duration necessary to detect CPE. Cytopathic/cytotoxic effect is monitored using an Inverted Compound Microscope.

In cases when viral CPE is undetectable using Inverted Compound Microscope, additional immunostaining with virus specific antibodies can be performed.

Calculations:

Viral and toxicity titers are expressed as −log₁₀ of the 50% titration end point for infectivity. To calculate the viral titer, a 50% tissue culture infectious dose (TCID₅₀) calculation—the Quantal test (Spearman-Karber Method)—will be applied.

Log TCID_{SO}=L−d(s−0.5)

Where:
L=−log₁₀ of the lowest dilution;
D=difference between dilution steps;
s=sum of proportions of positive wells.

The log₁₀ of infectivity reduction will be calculated as follows:

Log₁₀ Reduction Formula:

Log₁₀ Reduction=(log₁₀ TCID₅₀ of the Virus Control)−(log₁₀ TCID₅₀ of the Virucidal Suspension Test)

The percent reduction will be calculated as follows:

$$\% Reduction = [ 1 - \frac{TCI {D}_{50 } test}{TCI {D}_{50} baseline} ] \times 100$$

Test Product:

| | |
|---|---|
| Solution A: | |
| Active Ingredient: | Zinc Chloride, Ascorbic Acid |
| Solution B: | |
| Active Ingredient: | Sodium Chlorite |

Challenge Viral Strains:
Human Coronavirus (Betacoronavirus), strain OC43 (ZeptoMetrix Corp. #0810024CF)
Influenza A H1N1, strain A/WS/33 (ATCC #VR-1520)
Simian virus 40 (SV-40) (ATCC #VR-820)

Host Cells:
HCT-8 (ATCC #CCL-244; human colon adenocarcinoma, epithelial).
Madin-Darby Canine Kidney (MOCK) (ATCC #CCL-34; Madin-Darby Canine
Kidney cells)
BS-C-1 (ATCC #CCL-26; Cercopithecus aethiops [Green monkey] kidney cells)

Media:

The growth media and diluting fluids used in this study are as described in the Study Protocol in Addendum 1 of this Final Report.

Host Cell Preparation:

Cells, obtained from American Type Culture Collection (ATCC), were maintained as monolayers in disposable cell culture labware in accordance with BSLI SOP L-2084, “Procedure for Subculturing of Cells.” Prior to testing, host cell cultures were seeded onto 24-well cell culture treated plates. Cell monolayers for HCT-8 and BS-C-1 cells were no more than 48-hours old and 80-90% confluent before inoculation with the virus. Cell monolayers for MDCK cells were 49- to 52-hours old and 90% confluent before inoculation with the virus (See Deviation 02 in Addendum 1). The growth medium (GM) and maintenance medium (MM) used were RPMI-1640, MEM, or EMEM, as appropriate for each cell culture as summarized in Table 1.

TABLE 1
Host Cells and Media

| | | Growth | |
|---|---|---|---|
| | Host | Medium | Maintenance |
| Virus | Cell | Components | Medium Components |
| Coronavirus | HCT-8 | RPMI-1640 | RPMI-1640 |
| strain | | 10% Horse Serum | 2% Horse Serum |
| OC413 | | 1% Antibiotic | 1% Antibiotic |
| Influenza | MDCK | MEM | MEM |
| A H1N1 | | 10% FBS | 1% Antibiotic |
| | | 1% Antibiotic | 1% L-glutamine |
| | | 1% L-glutamine | ~1 μg/mL TPCK treated |
| | | | Trypsin |
| SV-40 | BS-C-1 | EMEM | EMEM |
| | | 10% FBS | 2% FBS |
| | | 1% Antibiotic | 1% Antibiotic |

Test Virus Preparation:

Test viruses used for this study were from BSLI virus stock. On the day of use, aliquots of a stock virus were removed from a −70° C. freezer and thawed prior to use in testing.

Test Product Preparation:

Prior to testing, the solutions A and B were stored in separate containers. To prepare the Test Product, the solutions A and B were combined in a 1:1 ratio in any appropriate volume immediately before use in testing. The combined solutions were shaken for a few seconds (before dispensing, and after combining) to mix the aqueous, soluble solutions. The individual solutions were exposed to air. They were re-capped for future use until the expiration date. Once combined, the Test Product was used in testing within 5 minutes of initial mixing.

Virucidal Suspension Test:

The Virucidal Suspension Test included the following parameters (Table 2):

TABLE 2
Parameters of Virucidal Suspension Test

| Parameter | Summary | Plating Replicates |
|---|---|---|
| Virucidal | 1 Part Virus + 9 Parts Test Product → Exposure → | 4 per dilution |
| Suspension Test | Neutralization → Dilution → Plating | |
| Virus Control | 1 part Virus + 9 Parts Diluent → Exposure → Dilution → | 4 per dilution |
| | Plating | |
| Cytotoxicity | Diluent + Test Product → Neutralization → Dilution → | 4 per dilution |
| Control | Plating | |
| Neutralization | 1 Part Diluent + 9 Parts Test Product → Neutralization → | 4 per dilution |
| Control | Virus inoculation | |
| Neutralizer | Virus + Diluent → Neutralization → Dilution → Plating | 4 per dilution |
| Toxicity Control | | |
| Cell Culture | Maintenance Medium | Minimum 4 wells |
| Control | | |

Deviations:

Two deviations from the Study Protocol occurred and are described below.

Deviation 01: Section 8.3 of the Study Protocol states that “Simian virus 40 (SV-40) (ATCC #VR-239)” will be used in this study. ATCC #VR-820 was the actual strain used in testing on 07/28/2020. The deviation was due to a typographical error. There was not an adverse effect on the outcome of the study. The virus successfully replicated in the Virus Control samples (TCID₅₀ of 6.75 log₁₀ in 1-hour control and 6.50 log₁₀ in the 4-hour control).

Deviation 02: Section 13.0 HOST CELL PREPARATION, of the Study Protocol states “ . . . Prior to testing, host cell cultures will be seeded onto 24-well cell culture treated plates. Cell monolayers will be more than 48-hours old and 80-90% confluent before inoculation with the virus . . . ” Section 13 0.0 should have stated that “Cell monolayers will not be more than 48-hours old . . . ” The Madin-Darby Canine Kidney (MDCK) (ATCC #CCL-34) cells used in testing versus Influenza A HINI on 08/11/2020 were incubated for greater that 48 hours. Actual incubation times are included in Table 3.

TABLE 3
Plate Incubation Details

| # of Plates & Time | # of Plates & Time | |
|---|---|---|
| Logged into | Logged Out of | |
| Incubator #061114 | Incubator #061114 | Samples Plated |
| 7/24-well plates | 5/24-well plates | 1-hour Virus Control |
| 10:45 on | 11:57 on Aug. 11, 2020 | 1-hour Test |
| Aug. 9, 2020 | Total incubation time: 49 h 12 m. | Neutralization Control |
| | | Neutralization |
| | | Toxicity Control |
| | | Cytotoxicity Control |
| | | Cell Control |
| | 2/24-well plates | 4-hour Virus Control |
| | 14:41 on Aug. 11, 2020 | 4-hour Test |
| | Total incubation time: 51 h 56 in. | |

Following being split at a 1:4 ratio, MDCK have taken closer to 48 hours to reach 80-90% confluence. Based on this information, the study director requested that the 24-well MDCK plates be prepared two days prior to testing instead of one. The deviation occurred because the study director did not specifically request that the cells be split in the afternoon of 08/09/2020 to avoid a deviation on 08/11/2020. There was not an adverse effect on the outcome of the study. The cells were still 90% confluent when used in testing on 08/11/2020 and the virus was successfully replicated in the Virus Control samples (TCID₅₀ of 7.50 log₁₀ in 1-hour control and 7.25 log₁₀ in the 4-hour control).

Results—Tables 4 Through 6:

Table 4 presents CPE, TCID₅₀ (log₁₀), log₁₀ reduction, and percent reduction data for the neutralization assay and test involving Human Coronavirus strain OC43 at two exposure times (1 hour and 4 hours). The neutralization was performed to verify that DIE neutralization broth was an effective neutralizer of Test Product, Solution A, combined with Solution B.

TABLE 4
Human Coronavirus strain OC43 Results
Test Product: Solution A (Zinc Chloride and Ascorbic Acid),
Combined with Solution B, (Sodium Chlorite)
Virus: Human Coronavirus OC43 ZeptoMetrix #: 0810024CF
Host Cell Line: HCT-8 Host Cell Line ATCC #: CCL-244
Volume Plated per Well (mL): 1.0 mL Neutralizer: DIE Neutralizing Broth

| | Virus | Virus | | | Neutralization | | | |
|---|---|---|---|---|---|---|---|---|
| Dilution | Control | Control | | Neutralization | Toxicity | Test | Test | |
| (−Log₁₀) | 1 Hour | 4 Hours | CTC | Control | Control | 1 Hour | 4 Hours | cc |
| | | | | | | | | 0000 |
| −2 | NT | NT | ++++ | CT | ++++ | CT | CT | N/A |
| −3 | NT | NT | 0000 | ++++ | ++++ | 0000 | 0000 | |
| −4 | ++++ | ++++ | 0000 | ++++ | ++++ | 0000 | 0000 | |
| −5 | ++++ | ++++ | NT | +++0 | ++++ | 0000 | 0000 | |
| −6 | ++++ | ++++ | NT | +++0 | 0+++ | 0000 | 0000 | |
| −7 | 0000 | 0000 | NT | 0000 | 000+ | 0000 | 0000 | |
| −8 | 0000 | 0000 | NT | NT | NT | NT | NT | |
| TCID₅₀ | 6.50 | 6.50 | 2.50 | 6.00 | 6.50 | <2.50 | <2.50 | |
| (log₁₀) | | | | | | | | |

| | | |
|---|---|---|
| Log10 Reduction | >4.00 | >4.00 |
| Percent Reductions | >99.99% | >99.99% |

+ CPE cytopathiclcytotoxic effect) present
0 CPE not detected
NT Not tested
CT Cytotoxicity
NA Not applicable
CC Cell Culture Control
TCIDso = L − d (s − 0.5)
L = −Log,o of the lowest plated dilution
d = Difference between dilution steps
s = Sum of proportions of positive wells
Log₁₀ Reduction = Log₁₀ Baseline − Log₁₀ Test
$\% Reduction = [ 1 - \frac{{TCID}_{50} test}{{TCID}_{50} baseline} ] \times 100$

Table 5 presents CPE, TCID₅₀ (log₁₀) reduction, and percent reduction data for the neutralization assay and test involving Influenza A strain A/WS/33 at the two exposure times (1 hour and 4 hours). The neutralization was performed to verify that D/E neutralization broth was an effective neutralizer of Test Product, (Solution A, [Zinc Chloride and Ascorbic Acid], combined with Solution B, [Sodium Chlorite]).

TABLE 5
Influenza A H1N1 strain A/WS/33 Results
Test Product: Solution A, (Zinc Chloride and Ascorbic Acid
Combined with Solution B, (Sodium Chlorite)
Virus: Influenza A H1N1 strain A/WS/33 ATCC# VR-1520
Host Cell Line: MDCK Host Cell Line ATCC #: CCL-34
Volume Plated per Well (mL): 1.0 mL Neutralizer: D/E Neutralizing Broth

| | Virus | Virus | | | Neutralization | | | |
|---|---|---|---|---|---|---|---|---|
| Dilution | Control | Control | | Neutralization | Toxicity | Test | Test | |
| (−Log₁₀) | 1 Hour | 4 Hours | CTC | Control | Control | 1 Hour | 4 Hours | CC |
| | | | | | | | | 0000 |
| −2 | NT | NT | ++++ | NT | NT | CT | CT | NA |
| −3 | NT | NT | 0000 | ++++ | ++++ | 0000 | 0000 | |
| −4 | ++++ | ++++ | 0000 | ++++ | ++++ | 0000 | 0000 | |
| −5 | ++++ | ++++ | NT | ++++ | ++++ | 0000 | 0000 | |
| −6 | ++++ | ++++ | NT | ++++ | ++++ | 0000 | 0000 | |
| −7 | +0++ | 0+++ | NT | 000+ | +++0 | 0000 | 0000 | |
| −8 | 0+00 | 0000 | NT | 0000 | 0000 | NT | NT | |
| TCID₅₀ | 7.50 | 7.25 | 2.50 | 6.75 | 7.25 | <2.50 | <2.50 | |
| (log₁₀) | | | | | | | | |

| | | |
|---|---|---|
| Log₁₀ Reduction | >5.00 | >4.75 |
| Percent Reductions | >99.99% | >99.99% |

Table 6 presents CPE, TCID₅₀ (log₁₀), log₁₀ reduction, and percent reduction data for the neutralization assay and test involving Simian virus 40 (SV-40) at two exposure times (1 hour and 4 hours). The neutralization was performed to verify that D/E neutralization broth was an effective neutralizer of Test Product, (Solution A, [Zinc Chloride and Ascorbic Acid], combined with Solution B, [Sodium Chlorite, Lot #CZ004B]).

TABLE 6
Simian virus 40 (SV-40) Results
Test Product: Solution A, (Zinc Chloride and Ascorbic Acid),
Combined with Solution B, (Sodium Chlorite)
Virus: Simian virus 40 (SV-40) ATCC# VR-820
Host Cell Line: BS-C-1 Host Cell Line ATCC #: CCL-26
Volume Plated per Well (mL): 1.0 mL Neutralizer: DIE Neutralizing Broth

| | Virus | Virus | | | Neutralization | | | |
|---|---|---|---|---|---|---|---|---|
| Dilution | Control | Control | | Neutralization | Toxicity | Test | Test | |
| (−Log₁₀) | 1 Hour | 4 Hours | CTC | Control | Control | 1 Hour | 4 Hours | CC |
| | | | | | | | | 0000 |
| −2 | NT | NT | ++++ | CT | ++++ | CT | CT | N/A |
| −3 | NT | NT | 0000 | ++++ | ++++ | ++++ | ++++ | |
| −4 | ++++ | ++++ | 0000 | ++++ | ++++ | ++++ | 000+ | |
| −5 | ++++ | ++++ | NT | ++++ | ++++ | ++++ | 0000 | |
| −6 | ++0+ | +0++ | NT | 0+++ | ++++ | ++0+ | 0000 | |
| −7 | 0++0 | 00+0 | NT | 0+00 | 0+0+ | 00+0 | 0000 | |
| −8 | 0000 | 0000 | NT | NT | NT | NT | NT | |
| TCID₅₀ | 6.75 | 6.50 | 2.50 | 6.50 | 7.00 | 6.50 | 3.75 | |
| (log₁₀) | | | | | | | | |

| | | |
|---|---|---|
| Log₁₀ Reduction | 0.25 | 2.75 |
| Percent Reductions | 43.77% | 99.82% |

Study Conclusions:

Under the conditions of this evaluation, the Test Product, the combination of Solution A and Solution B, reduced the infectivity of Coronavirus strain OC43 by >4.00 log₁₀ (>99.99%) following both exposure times (1-hour and 4-hours).

Under the conditions of this evaluation, the Test Product, the combination of Solution A and Solution B, reduced the infectivity of Influenza A H1N1 strain A/WS/33 by >5.00 log₁₀ (>99.99%) following the 1-hour exposure time and by >4.75 log 10 (>99.99%) following the 4-hour exposure time.

Under the conditions of this evaluation, the Test Product, the combination of Solution A and Solution B, reduced the infectivity of Simian virus 40 (SV-40) by 0.25 log₁₀ (43.77%) following the 1-hour exposure time and by 2.75 log₁₀ (99.82%) following the 4-hour exposure time.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A method for treating a disease or disorder of the respiratory system of a mammal caused by the presence of a pathogen, the method comprising:
(a) supplying a two component aqueous therapeutic composition comprising a first component containing at least one transition metal in elemental, ionic, compound, or complexed form, dissolved and/or dispersed in an aqueous medium, and a second component containing an aqueous solution of an oxidation-reduction potential raising component, the oxidation-reduction potential raising component aiding the concentration of transition metal ions of the transition metal of the first component;
(b) aerosolizing the two components of the two component aqueous therapeutic composition, either separately or following admixture of the two components;
(c) introducing an aerosol formed in step (b) into the respiratory system of said mammal.

2. The method of claim 1, wherein the transition metal present in the first component is selected from the group consisting of zinc, silver, copper, cobalt, nickel, and mixtures thereof.

3. The method of claim 1, wherein the transition metal is present in aqueous solution having a pH of less than 7.

4. The method of claim 1, wherein the oxidation-reduction potential raising component is selected from the group consisting of hydrogen peroxide, chlorite, or a mixture thereof.

5. The method of claim 1, wherein the oxidation-reduction potential raising component comprises a soluble chlorite.

6. The method of claim 1, wherein the first component and the second component are separately stored, combined immediately prior to aerosolization, and aerosolized.

7. The method of claim 1, wherein a further active substance is present in one of the two components, or in a separate component, the active substance being selected from the group consisting of antimicrobials, vitamins, antivirals, and desensitizing agents.

8. The method of claim 1, wherein a mean droplet size of the aerosol is less than 3 μm.

9. The method of claim 1, wherein a mean droplet size of the aerosol is between 0.5 and 2.5 μm.

10. The method of claim 1, wherein at least one of the first component and second component further comprises ascorbic acid.

11. An aerosol suitable for treating a disease or disorder of a mammalian respiratory system, the aerosol containing droplets having a mean droplet size less than 3 μm, the droplets comprising a two component aqueous therapeutic composition as described in claim 1, each component of the aqueous therapeutic composition being contained in the same droplets and/or in separate droplets of the aerosol.

12. The aerosol of claim 10 further comprising one or more antibacterial, antiviral, vitamin, or desensitizing agent components, supplied as a separate component or in one or both of said first and second components.

13. The aerosol of claim 10, wherein the transition metal is zinc.

14. The aerosol of claim 12, wherein zinc is present in the form of Zn′ ions.

15. The aerosol of claim 10, wherein the oxidation-reduction potential raising component comprises chlorite ions.

16. The aerosol of claim 14, wherein the second component is maintained at a neutral or basic pH.

17. The aerosol of claim 10, the droplets further comprising ascorbic acid.
